(19) United States
(12) Patent Application Publication
(10) Pub. No.: US 2019/0201153 A1
(43) Pub. Date: Jul. 4, 2019

(54) URNIARY CATHETER CONNECTOR FOR IRRIGATION OF A CATHETERIZED BLADDER

(71) Applicant: MEDULY AB, Borås (SE)

(72) Inventors: Susanne HÄGGLUND, Nykvarn (SE); Andreas BÄCKÄNG, Äspered (SE); Julia SCHRÖDER, Äspered (SE)

(73) Assignee: Meduly AB, Borås (SE)

(21) Appl. No.: 16/325,266

(22) PCT Filed: Aug. 16, 2017
(86) PCT No.: PCT/EP2017/070769

§ 371 Date: Feb. 13, 2019

(30) Foreign Application Priority Data
Aug. 17, 2016 (EP) 1651108-1

(51) Int. Cl. A61B 90/00; A61M 3/02; A61M 25/00; A61M 39/24

(52) U.S. Cl. CPC A61B 90/05; A61M 3/0262; A61M 2039/1033; A61M 25/0097; A61M 39/24; A61M 25/0017

(57) ABSTRACT

A catheter connector including a body including a first end, a second end, an outer and an inner surface extending between the first and second ends, a first port arranged at the first end of the body and configured to receive a syringe, a second port for connection to a urinary catheter and arranged at the second end of the body, and a channel connecting the first and second ports, whereby the channel has two openings at said ports. The catheter connector includes a splash guard extending circumferentially from the outer surface of the body, wherein the first port is configured to receive the syringe on the inner surface of the body and the second port is configured to receive the urinary catheter on the outer surface of the body.

### FIELD OF THE INVENTION

The present invention relates to a urinary catheter connector for irrigation of a catheterized bladder. The urinary catheter connector includes a body comprising a first end, a second end, an outer and an inner surface extending between the first and second ends, a first port arranged at the first end of the body and configured to receive a syringe, a second port for connection to a urinary catheter and arranged at the second end of the body, and a channel connecting the first and second ports, whereby the channel consists of two openings at said ports.

### BACKGROUND OF THE INVENTION

A urinary catheter system is made up of a catheter inserted into a bladder and a urine collector device connected to the catheter and adapted to collect the urine from the bladder. The collecting device may for example be a drainage bag. Patients with clots, debris or viscous urine may need frequent irrigation of the bladder to prevent clogging of the catheter. For bladder irrigation purposes a piston syringe is often used. The syringe is filled with rinsing fluid and connected to the catheter. A specified amount of rinsing fluid is injected into the bladder and then aspirated from the bladder through the catheter using the syringe. The aspirated liquid is emptied in a container and the syringe is filled with clean rinsing fluid. This irrigation procedure is repeated until the liquid aspirated from the bladder is clear.

Typically, a health professional, such as a nurse, holds and clamps the end of the catheter to the syringe with one hand, while operating the syringe with the other hand. This means that, when emptying and filling the syringe, only one hand can be used. The other hand must hold and clamp the end of the catheter to prevent uncontrolled backflow of liquids from the catheter. Uncontrolled backflow of liquids from the catheter may result in accidental aspiration of more fluid from the bladder than has been injected, which in turn may damage the patient's bladder.

Leakage and spilling of urine is not uncommon during irrigation of the bladder and catheter. If the catheter becomes blocked, e.g. by blood clots, an overpressure builds inside the catheter, which may cause a leak in the connection between the syringe and the catheter. Leaks caused by overpressure frequently result in health professionals and the patient being sprayed with urine during bladder irrigation. The spraying, spilling and leakage of urine puts health professionals and patients at risk for bio-hazardous waste exposure, such as contamination by viruses, bacteria and the like.

Some urine catheter systems have been developed to prevent contamination by spraying and spilling of urine. Other systems have been developed to allow health professionals to use both hands during irrigation.

US2012/0232503 discloses a urinary catheter connector system comprising a body and three ports, a catheter port, an irrigation port and a urine exit port. A channel in the body connects the three ports. A valve is positioned in the body adapted to cooperate with an irrigating syringe to shut off a flow of fluid and air to the urine exit port when the irrigating syringe is inserted and allows for the flow of fluid and air to the urine exit port after removal of the syringe.

US2005/0194047 discloses a connector for a fluid administration system comprising a body or housing, a first and second conduit defined by the housing, which are in fluid communication, and a check valve disposed within the housing. The valve comprises a first and a second passageway in a first and second body portion, respectively. The first body portion is in receiving relation with the second body portion, whereby the second body portion is movable in relation to the first body portion in a first and second position. In the first position, a flow of fluid is allowed in one direction and in the second position, the flow is allowed in a second direction, which is opposite the first direction

U.S. Pat. No. 5,738,663 discloses a housing with a medical valve comprising a seal adapted to be in a compressed state upon insertion of e.g. a syringe and in a relaxed state after removal of the syringe. Channels defined by the housing are in fluid communication when the valve is in the compressed state.

U.S. Pat. No. 4,819,684 discloses a shut off valve adapted to be positioned in a fusion line comprising a body with passages to an inlet, an outlet and an orifice adapted for receiving a syringe. A deformable diaphragm inside the body separates the three passages inside the body. In case of clogging, an overpressure from the catheter causes spraying of urine through the catheter. This system does not prevent contamination risks for health professionals and patients.

US 2007/0173773 discloses a splash shield adapted for wound irrigation, i.e. minor amount of flowing fluids. The shield includes a dome-shaped shield with a channel through the top to receive a syringe. Air vents are present around the top channel for exchange of air but not fluids. This shield is not adapted for use with a catheter, for which a connector means for both syringe and catheter are needed.

WO2011/073916 discloses a shield for a medical tool, whereby the shield consists of a shield and an aperture. The aperture is adapted to receive a bushing in which a medical tool can be inserted. The shield comprises a concave inner surface and is bent inwards, towards a first port of the aperture.

### OBJECT AND SUMMARY OF THE INVENTION

It is an object of the present invention to at least partly overcome the above problems, and to provide an improved urinary catheter connector for bladder irrigation of a catheterized bladder.

This object is achieved by a catheter connector as defined in claim 1.

The present invention relates to a catheter connector including a body comprising or consisting of a first end, a second end, an outer and inner surface extending between the first and second ends, a first port arranged at the first end of the body and configured to receive a syringe, a second port for connection to a urinary catheter and arranged at the second end of the body, and a channel connecting the first and second ports, whereby the channel consists of two openings at said ports. The inner surface surrounds the channel. The catheter connector is characterized in that the catheter connector comprises a splash guard extending circumferentially from the outer surface of the body, wherein the first port is configured to receive the syringe on the inner surface of the body and the second port is configured to receive the urinary catheter on the outer surface of the body. The syringe and the urinary catheter come never in contact with each other.

In a further embodiment, a contact area around the second port, i.e. the outer surface for connecting the catheter and the body is less than a contact area around the first port and the channel, i.e. the inner surface of the channel where a syringe is inserted. This difference in contact area allows the friction between the syringe and the body to be larger than the friction between the catheter and the body. This ensures that, in case of over-pressure from the catheter, the flow of urine fluid will flow between the outer surface at the second port and the catheter and thus be intercepted by the splash guard. This prevents the flow of fluid to escape through the first port.

In one embodiment, the splash guard extending circumferentially from the outer surface of the body, towards the second end of the body and arranged so that a space for intercepting fluid from the catheter is formed between the splash guard and the outer surface of the body.

Because the splash guard surrounds the body of the connector, any spattering or spilling from an end of the catheter will be intercepted by the splash guard and will thus not be spattered over the health professional or the patient. This reduces the risk of bio-hazardous waste exposure, such as contamination by viruses, bacteria and the like. Further, the catheter connector can be handled single handed, which improves ergo dynamics and safety for health professionals. The working environment and safety of the health professional is thus improved by using the catheter connector according to the invention.

In an embodiment of the invention, the splash guard is bent inwards towards the second end of the body. In another embodiment, the splash guard has an inner surface facing the outer surface of the body, and the inner surface of the splash guard is concave. In a further embodiment, said space substantially has the shape of a toroid. In yet another embodiment, the splash guard is extending from the first end of the body towards the second end of the body. The geometry of the splash guard allows collection and aiming of urine that may be spilled during irrigation. In case of clogging, the pressure builds up in the catheter and the flow of urine may escape the catheter at high speed. The geometry of the splash guard reduces the speed of the flow and thereby further reduces the risk of contamination as explained above.

In an embodiment, the body and the splash guard are rotational symmetric, and the splash guard and the body are coaxially arranged. In another embodiment, the splash guard has an opening, and the body is extending through the opening of the splash guard. The splash guard may be removable attached to the body. The splash guard may be moved or slit over the body prior to use. The splash guard may be fixedly attached to the body. In one embodiment, a first end of the splash guard is attached to the body and a second end of the splash guard has an opening, and the body is extending through the opening of the splash guard.

The opening of the splash guard is preferably concentrically arranged in relation to the first port. The opening of the splash guard is preferably also concentrically arranged in relation to the second port. This concentrically arrangement reduces speed of the urine flow as explained above.

In a further embodiment, the splash guard is a part of the body. The splash guard may be a separate part of the catheter connector that can be attached or connected to the body prior to use. The body and the splash guard may also be made in one piece such that the splash guard is a part of the body.

In an embodiment, said body is conically shaped and is tapering towards the second end. The tapered shape allows for an easy connection with the catheter, which can be slit over the outer surface at the second end at the second port. The second port may be arranged at a distance from the splash guard. The distance may be between 1.5 and 8 cm, or between 2 and 6 cm, or between 2 and 5 cm. This distance improves convenience of use of the catheter connector when the catheter needs to be connected or slit over the second port.

In another embodiment of the invention, the body further comprises a check valve positioned in the channel. The valve prevents fluid from flowing out of the catheter when no syringe is inserted in the catheter connector. Because the valve stops the flow of fluid from the catheter, the health professional does not need the hold and clamp the catheter. The valve also enables the health professional to maintain control over the amount of liquid that has been injected into, and aspirated from the bladder. This improves both patient and work safety and further reduced contamination risks. It also improves the ergo-dynamics for the professional as both hands can be used for emptying and filling the syringe with rinsing fluid.

In an embodiment, the check valve comprises a membrane adapted to seal the channel and adapted to open the channel and thus to allow a flow through the channel upon insertion of a syringe through the irrigation port. In one embodiment, a two way flow through the channel is allowed upon insertion of the syringe. A flexible membrane can be mounted in the channel. The membrane may be pushed open upon insertion of the syringe through the first port and resumes its position of sealing the channel after removal of the syringe, i.e. a check valve.

In another embodiment, the check valve is positioned in the first port. The valve may be positioned anywhere in the channel where the syringe can open the valve. Manufacturing of the catheter connector is less complicated if the valve is positioned in the first port.

In an embodiment, the first port comprises a first attachment member for connecting the first port and the syringe, whereby the first attachment member is selected from threads, mating indentation and protrusion and a Luer-lock, and the like. The attachment member may be a Luer-lock. Syringes with Luer lock are readily available. A Luer lock fitting can be made on the first port.

In another embodiment, the second port comprises a second attachment member for connecting the outer surface of the body in the proximity of the second port and the urinary catheter, whereby the second attachment member is selected from threads, mating indentation and protrusion, and the like.

The first and second attachment member may be a thread, such that the syringe can be screwed in the first port. As variations on full screwing threads, different types of partly threaded or clip members may be used. Alternatively, the first port may have an attachment indentation and the syringe an attachment protrusion, whereby the attachment indentation and the attachment protrusion can be mated and snapped together to secure the syringe to the connector. In one embodiment, the catheter connector does not have a check valve. If attachment members are used that prevent the urinary catheter from becoming detached from the catheter connector i.e. in case of overpressure, no check valve is needed.

In a further embodiment, the catheter connector comprises a first cap adapted to be connected to the first or second port using an attachment member at one end of the cap, and adapted to be connected to the syringe or urinary catheter using the attachment member at a second end of the cap. The cap may for example have a thread on one end of the cap to screw the cap to the connector and a Luer Lock on the other end to connect the syringe to the cap.

The invention also relates to a catheter connector including a body comprising a first end, a second end, an outer and an inner surface extending between the first and second ends, a first port arranged at the first end of the body and configured to receive a syringe, a second port for connection to a urinary catheter and arranged at the second end of the body, and a channel connecting the first and second ports, whereby the channel consists of two openings at said ports characterized in that the catheter connector comprises a splash guard extending circumferentially from the outer surface of the body,

wherein the attachment member comprises an annular groove in a wall of the body extending from the first end toward the second end or vice versa and forming an opening adapted to receive the urinary catheter or the syringe, respectively.

In one embodiment, the attachment member comprises a second cap adapted to be connected to the second and/or first port using the attachment member.

The second cap may be fitted over the urinary catheter prior to connecting the urinary connector to the catheter connector, such that the cap can be moved over the attachment member that connects the urinary catheter with the catheter connector, and then can be attached to the outer surface of the body of the connector at the second port. e.g. by screwing the cap onto the outer surface at the second port. A similar second cap can be used to connect the syringe to the catheter connector.

### BRIEF DESCRIPTION OF THE DRAWINGS

The invention will now be explained more closely by the description of different embodiments of the invention and with reference to the appended figures.

FIG. 1 shows a side view of a catheter connector.

FIG. 2 shows a perspective view of a front side of the catheter connector.

FIG. 3 shows a cross section of a catheter connector.

FIG. 4 shows a perspective view of a back side of the catheter connector.

FIG. 5 shows a cross section of the catheter connector connected to a catheter.

FIG. 6 shows a cross section of the connector in FIG. 6 after insertion of a syringe.

FIG. 7 illustrates flows of urine fluid through the catheter connector.

FIG. 8 shows a cross section of a second embodiment of the invention.

FIG. 9 shows a perspective view of a third embodiment of the invention.

### DETAILED DESCRIPTION OF PREFERRED EMBODIMENTS OF THE INVENTION

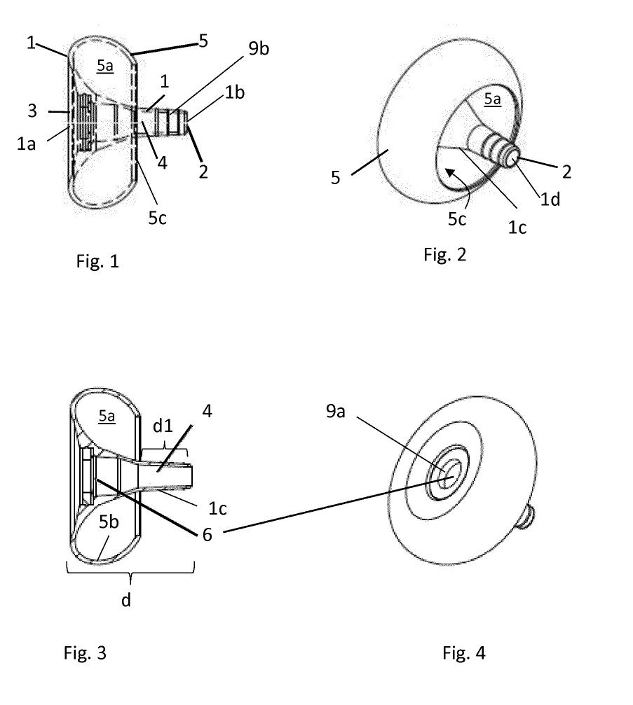

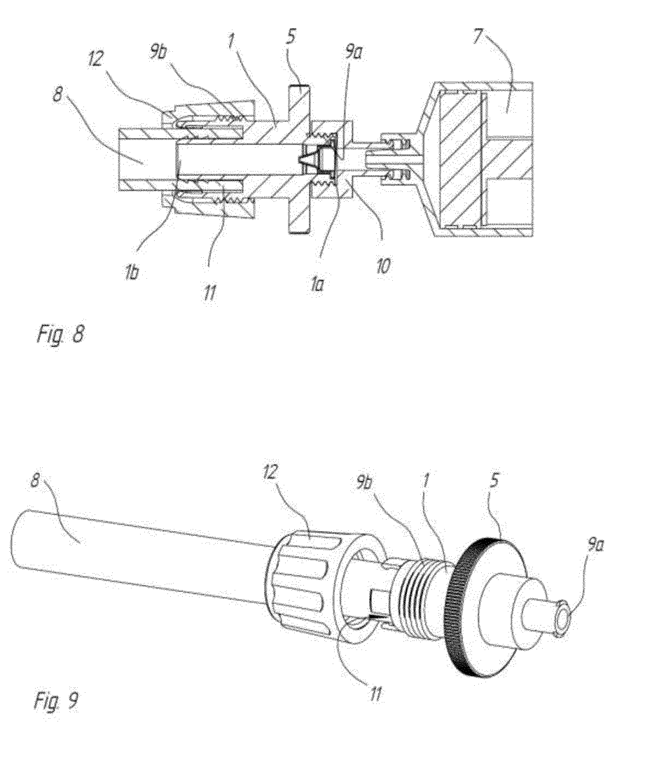

FIG. 1 shows a catheter connector. The connector includes a body 1 comprising a first end 1a and a second end 1b. An outer surface 1c and an inner surface 1d extends between the first and second ends. The body may be conically shaped and be tapering towards the second end 1b as shown in FIGS. 1 to 4. The body may be straight or rectangular shaped as shown in FIGS. 8 and 9.

The body 1 consist of two openings at a first port 3 and at a second port 2. The first port 3 is arranged at the first end 1a of the body. This first port 3 is configured to receive a syringe 7 as shown in FIGS. 6 and 7 on the inner surface 1d of the body (1). The first port 3 may comprise a first attachment member 9a for connecting the first port and the syringe 7. The first attachment member 9a may be selected from full or partial threads, mating indentation and protrusion and a Luer-lock. The first port 3 may be formed with one or more indentation adapted to receive a portion 7b of the syringe 7.

A second port 2 is configured to connect to a urinary catheter 8 on the outer surface 1c of the body (1). The second port 2 is arranged at the second end 1b of the body 1 and may be arranged at a distance d1 from a splash guard 5 as shown in FIG. 3. The distance d1 may be between 1.5 and 8 cm, or between 2 and 6 cm, or between 2 and 4 cm. The catheter 8 may be slit over the outer surface of the catheter connector as shown in FIGS. 6 and 7. The diameter of the body 1 at the second end 2 is adapted to receive the catheter 8 and to provide a tight fit of the catheter over the body. A contact area C1 as shown in FIG. 6 preferably has at least a length of d1 to ensure that a splashing flow of urine F2 is intercepted by the splash guard 5 as shown in FIG. 7. The second port 2 comprises a second attachment member 9b for connecting the outer surface of the body 1 in the proximity of the second port 2 and the urinary catheter 8. The second attachment member 9b may be selected from threads and mating indentation and protrusion and other known alternatives.

The catheter connector may comprise a first cap 10. The cap is adapted to be connected to the first port 3 using attachment member 9a at one end of the cap 10, and adapted to be connected to the syringe 7 using the attachment member 9a at a second end of the cap. Likewise, the catheter connector may comprise a first cap 10 adapted to be connected to the second port 2 using attachment member 9b at one end of the cap, and adapted to be connected to the urinary catheter 8 using the attachment member 9b at a second end of the cap.

The first port 3 and the second port 2 are connected by a channel 4 as shown in FIG. 3. The channel has only two openings at each end 1a, 1b. The length of the channel may vary and depend on the length of the tip of the syringe. The tip of the syringe is to be inserted into the channel 4. Therefore, the diameter of the first port 3 and the channel are adapted to receive the syringe and to provide a tight fit for the syringe into the catheter connector. A contact area C2 as shown in FIG. 6 preferably has at least a length of 50% of the channel, or a length between 50 and 100% of the channel. The contact area C2 may have a maximum contact length equivalent to the length of channel 4. When the first cap 10 is used to connect the syringe to the first port, the contact area C2 can be less than 50%, or 25%. This is because the cap extends the contact area C1 inside the first cap 10.

The contact area C1 around the second port 2, i.e. the contact area between the catheter 8 and the body 1 is less than the contact area C2 around the first port 3 and the channel, i.e. the contact area between the tip of the syringe 7a and the body 1. This allows the friction between the syringe 7 and the body 1 to be larger than the friction between the catheter 8 and the body 1.

Alternatively, as shown in FIGS. 8 and 9, the contact areas C1, C2 are a portion of the length of the body in case the attachment members 9a and 9b are screwing threads.

The catheter connector comprises a splash guard 5 extending circumferentially from the outer surface 1c of the body towards the second end 1b of the body 1. The splash guard may be arranged so that a space 5a is formed for intercepting fluid from the catheter 8 between the splash guard and the outer surface of the body 1 as indicated by the arrowed line F2 in FIG. 7.

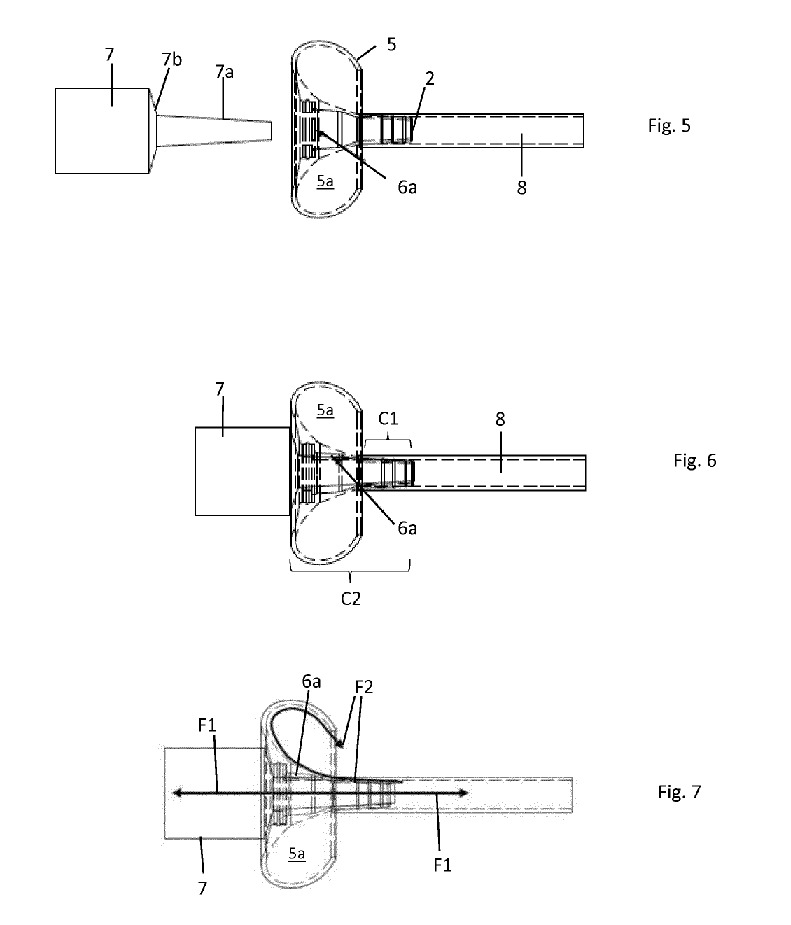

As shown in FIGS. 2 and 4, the splash guard 5 surrounds at least a portion of the body and extends from the first end of the body towards the second end of the body. From the first end 1a, the splash guard 5 bents outwardly in a direction away from the first port 3 and subsequently bents inwards towards the second end 1b of the body 1. The splash guard has an inner surface 5b partly facing the outer surface 1c of the body. The inner surface of the splash guard is concavely formed as shown in FIGS. 1 to 7. The space 5a formed by the splash guard is preferably substantially toroidal shaped. Preferably, the body 1 and the splash guard 5 are rotationally symmetric, and the splash guard 5 and the body are coaxially arranged. As shown in FIG. 4, a first end of the splash guard 5 is attached to the body 1 and a second end of the splash guard has an opening 5c. The body extends preferably through the opening of the splash guard as shown in the FIGS. 1 to 7.

The opening 5c of the splash guard 5 is preferably concentrically arranged in relation to the first port 3 and in relation to the second port 2.

The splash guard 5 may be a part of the body. The splash guard may also be a connectable part of the catheter connector, which can be connected to the catheter connector prior to use. The splash guard may also be slit over the body 1 prior to connecting the syringe and the catheter. The body 1 and the splash guard a5 may be made in one piece of material as shown in FIG. 1.

The catheter connector according to the invention may further comprise a check valve 6 positioned in the channel 4. The check valve 6 may be positioned in the first port 3. Many different types of valves may be used for sealing the channel of the catheter connector. FIGS. 6 and 7 show a membrane 6a. This membrane is adapted to open the channel upon insertion of a syringe 7 through the first port 3 as shown in FIGS. 6 and 7. The membrane 6a is furthermore adapted to seal the channel 4 after removal of the syringe 7. In one embodiment, a catheter connector with a check valve is disclaimed.

The catheter connector may be made of different types of materials with different properties, such as, but not limited to, plastics, metals or bio based materials, such as biodegradable. The materials may need to be sterilizable. The catheter connector can be designed to be a disposable product or a re-usable product. One example of biodegradable material is biodolomer.

In one aspect of the invention, the catheter connector includes a body 1a first port 3, a second port 2, a channel 4 and a splash guard 5 as outlined above. The catheter connector comprises the attachment member (9a, 9b) comprises an annular groove 11 in a wall of the body 1 extending from the first end toward the second end or vice versa and forming an opening 11 adapted to receive the urinary catheter 8 or the syringe 7, respectively.

As shown in FIG. 8, the catheter connector comprises attachment member 9b, which comprises an annular groove 11 in a wall of the body 1 extending from the second end 1b toward the first end 1a thus forming an opening 11 adapted to receive the urinary catheter 8.

The attachment member 9a, 9b may comprise a second cap 12 adapted to be connected to the second and/or first port 2, 3 using the attachment member 9a, 9b. In FIGS. 8 and 9, a cap 12 is shown at the second end of the body. The second port has attachment member 9b comprising a groove 11 and a screw thread 9b. The cap 12 is first moved over the urinary catheter after which the catheter 8 can be positioned in the annual groove 11 and then screwed on the second port 2 using the screwing threads.

The present invention is not limited to the embodiments disclosed but may be varied and modified within the scope of the following claims. For example, the body 1 and the splash guard 5 may be asymmetrically or rotational asymmetrically in relation to each other.

1. A catheter connector including a body comprising a first end, a second end, an outer and an inner surface extending between the first and second ends, a first port arranged at the first end of the body and configured to receive a syringe, a second port for connection to a urinary catheter and arranged at the second end of the body, and a channel connecting the first and second ports, whereby the channel consists of two openings at said ports, wherein the catheter connector comprises a splash guard extending circumferentially from the outer surface of the body, wherein the first port is configured to receive the syringe on the inner surface of the body and the second port is configured to receive the urinary catheter on the outer surface of the body.

2. The catheter connector according to claim 1, wherein the splash guard extends circumferentially from the outer surface of the body, towards the second end of the body and arranged so that a space for intercepting fluid from the catheter is formed between the splash guard and the outer surface of the body.

3. The catheter connector according to claim 1, wherein the splash guard is bent inwards towards the second end of the body.

4. The catheter connector according to claim 1, wherein the splash guard has an inner surface facing the outer surface of the body, and the inner surface of the splash guard is concave.

5. The catheter connector according to claim 1, wherein said space substantially has the shape of a toroid.

6. The catheter connector according to claim 1, wherein the splash guard is extending from the first end of the body towards the second end of the body.

7. The catheter connector according to claim 1, wherein the body and the splash guard are rotational symmetric, and the splash guard and the body are coaxially arranged.

8. The catheter connector according to claim 1, wherein the splash guard is a part of the body.

9. The catheter connector according to claim 1, wherein the body further comprises a check valve positioned in the channel.

10. The catheter connector according to claim 9, wherein the check valve comprises a membrane adapted to seal the channel and adapted to allow a flow through the channel upon insertion of a syringe through the irrigation port.

11. The catheter connector according to claim 9, wherein the check valve is positioned in the first port.

12. The catheter connector according to claim 1, wherein the first port comprises a first attachment member for connecting the first port and the syringe, whereby the first attachment member is selected from threads, mating indentation and protrusion and a Luer-lock.

13. The catheter connector according to claim 1, wherein the second port comprises a second attachment member for connecting the outer surface of the body in the proximity of the second port and the urinary catheter, whereby the second attachment member is selected from threads and mating indentation and protrusion.

14. The catheter connector according to claim 12, wherein the catheter connector comprises a first cap adapted to be connected to the first or second port using attachment member at one end of the cap, and adapted to be connected to the syringe or urinary catheter using the attachment member at a second end of the cap.

15. A catheter connector including a body comprising a first end, a second end, an outer and an inner surface extending between the first and second ends, a first port arranged at the first end of the body and configured to receive a syringe, a second port for connection to a urinary catheter and arranged at the second end of the body, and a channel connecting the first and second ports, whereby the channel consists of two openings at said ports, wherein the catheter connector comprises a splash guard extending circumferentially from the outer surface of the body,
wherein the attachment member comprises an annular groove in a wall of the body extending from the first end toward the second end or vice versa and forming an opening adapted to receive the urinary catheter or the syringe, respectively.

16. The catheter connector according to claim 15, wherein the attachment member comprises a second cap adapted to be connected to the second and/or first port using the attachment member.
